(19) United States
(12) Patent Application Publication
(10) Pub. No.: US 2026/0077235 A1
(43) Pub. Date: Mar. 19, 2026

(54) EXERCISE TRAINING PLAN GENERATION METHOD AND APPARATUS, DEVICE, AND STORAGE MEDIUM

(71) Applicant: GUANGDONG COROS SPORTS TECHNOLOGY CO., LTD, Dongguan (CN)

(72) Inventors: Yang YANG, Dongguan (CN); Naixi LIU, Dongguan (CN); Xin LIU, Dongguan (CN)

(21) Appl. No.: 18/884,593

(22) Filed: Sep. 13, 2024

(51) Int. Cl. A63B 24/00

(52) U.S. Cl. CPC A63B 24/0075; A63B 2024/0065

(57) ABSTRACT

A method includes obtaining an exercise state of a user, determining a current physical strength of the user according to the exercise state, and generating an exercise training plan according to the current physical strength. The exercise state includes at least one of an aerobic exercise state or an anaerobic exercise state.

### TECHNICAL FIELD

The present disclosure generally relates to the sports technology field and, more particularly, to an exercise training plan generation method and apparatus, a device, and a storage medium.

### BACKGROUND

With the improvement in living standards, health has also become increasingly important for people, and more people have started to exercise regularly to stay healthy. Smart wearable devices are widely used in sports activities, including track and field, and marathons. Due to lightweight design and functions such as positioning, navigation, and training guidance, smart wearable devices have gained popularity among people doing sports. Physical strength is an indication of evaluating the current physical strength state of the body. A user can be guided to perform training with corresponding loads and provided with a training plan with the corresponding loads according to the physical strength. Currently, the existing wearable devices are not able to accurately calculate the physical strength of the user and are not able to generate a corresponding training plan based on the physical strength.

### SUMMARY

In accordance with the disclosure, there is provided a method. The method includes obtaining an exercise state of a user, determining a current physical strength of the user according to the exercise state, and generating an exercise training plan according to the current physical strength. The exercise state includes at least one of an aerobic exercise state or an anaerobic exercise state.

In accordance with the disclosure, there is provided an electronic device, including at least one processor and at least one memory. The at least one memory stores at least one computer program that, when executed by the at least one processor, causes the at least one processor to obtain an exercise state of a user, determine a current physical strength of the user according to the exercise state, and generate an exercise training plan according to the current physical strength. The exercise state includes at least one of an aerobic exercise state or an anaerobic exercise state.

In accordance with the disclosure, there is provided a computer-readable storage medium storing at least one computer program that, when executed by at least one processor, causes the at least one processors to obtain an exercise state of a user, determine a current physical strength of the user according to the exercise state, and generate an exercise training plan according to the current physical strength. The exercise state includes at least one of an aerobic exercise state or an anaerobic exercise state.

### BRIEF DESCRIPTION OF THE DRAWINGS

FIG. 1 is a schematic flowchart of an exercise training plan generation method according to some embodiments of the present disclosure.

FIG. 2 is a schematic flowchart of obtaining an exercise state of a user in an exercise training plan generation method according to some embodiments of the present disclosure.

FIG. 3 is a schematic flowchart of determining current physical strength of a user in an exercise training plan generation method according to some embodiments of the present disclosure.

FIG. 4 is a schematic flowchart of adjusting a physical strength condition in an exercise training plan generation method according to some embodiments of the present disclosure.

FIG. 5 is a schematic flowchart of generating an exercise training plan in an exercise training plan generation method according to some embodiments of the present disclosure.

FIG. 6 is a schematic structural block diagram of an electronic device according to some embodiments of the present disclosure.

### DETAILED DESCRIPTION OF THE EMBODIMENTS

To make the objectives, technical solutions, and advantages of the present disclosure clearer, the present disclosure is described in detail in connection with the accompanying drawings and embodiments of the present disclosure. Described embodiments are merely used to describe the present disclosure and not limit the present disclosure.

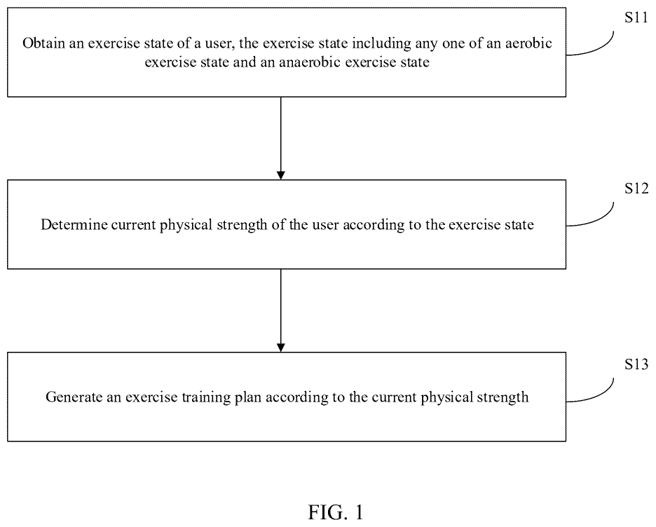

FIG. 1 is a schematic flowchart of an exercise training plan generation method according to some embodiments of the present disclosure. In some embodiments, the method includes processes S11 to S13.

At S11, an exercise state of a user is obtained. The exercise state includes any one of an aerobic exercise state and an anaerobic exercise state.

In some embodiments, the aerobic exercise state and the anaerobic exercise state can be two different exercise states in exercise physiology. The aerobic exercise state can refer to a state in which a human body exercises with sufficient oxygen supply, while the anaerobic exercise state can refer to a state in which body muscles exercise rapidly and intensely with insufficient oxygen. The aerobic exercise can be an exercise with low intensity, low frequency, and long duration, and a heart rate can reach 120 to 150 beats per minute. The anaerobic exercise can be an exercise with high-intensity, high-frequency, and short duration, and the heart rate can reach 170 to 180 beats per minute or above.

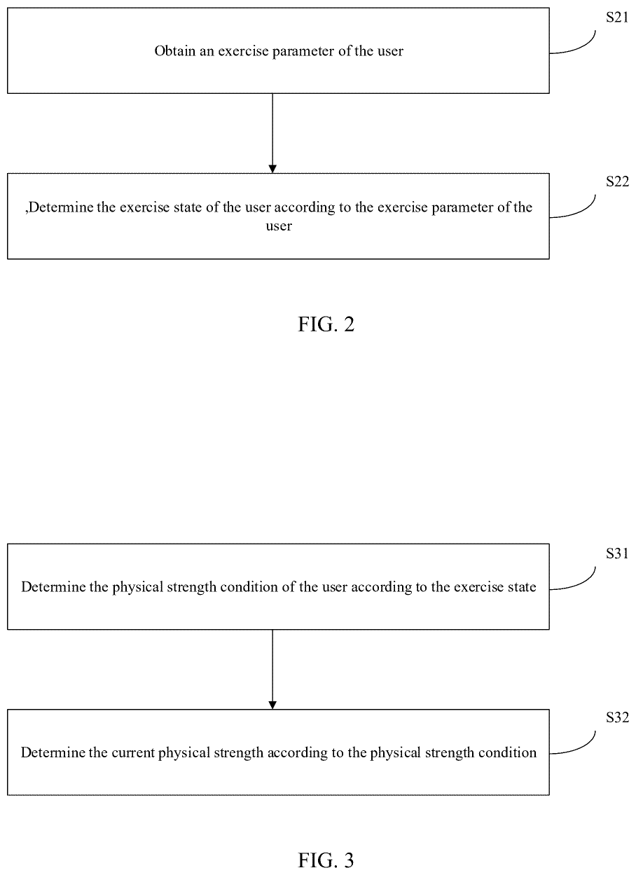

In some embodiments, FIG. 2 is a schematic flowchart showing obtaining the exercise state of the user in the exercise training plan generation method according to some embodiments of the present disclosure.

At S21, an exercise parameter of the user is obtained.

At S22, the exercise state of the user is determined according to the exercise parameter of the user.

In some embodiments, the exercise parameter of the user can be obtained. Then, the exercise state of the user can be determined according to the exercise parameter of the user. The exercise parameter can be measured using a wearable device, e.g., a sport watch, carried by the user, such as using a sensor built in the wearable device. For example, when the user is running, a running parameter of the user can include but is not limited to a heart rate parameter, a heart rate variability parameter, a pace parameter, and an acceleration parameter. According to the exercise parameter, whether the exercise of the user belongs to an exercise of low-intensity, low-frequency, and long duration, with a heart rate between 120 to 150 beats per minute, or belongs to an exercise of high-intensity, high-frequency, and short duration, and with the heart rate above 170 to 180 beats per minute can be analyzed. If the exercise of the user belongs to the exercise of low-intensity, low-frequency, and long duration, and with the heart rate between 120 to 150 beats per minute, the exercise state of the user can be determined as the aerobic exercise state. If the exercise of the user belongs to the exercise of low-intensity, low-frequency, and long duration, and with the heart rate above 170 to 180 beats per minute, the exercise state of the user can be determined as the anaerobic exercise state. The heart rate can be measured using a wearable device, e.g., a sport watch, carried by the user, such as using a heart rate sensor built in the wearable device.

In some embodiments, an exercise parameter threshold can be set to be a basis for determining the aerobic exercise state and the anaerobic exercise state. For example, an exercise parameter threshold correspondingly included in an aerobic exercise condition and an exercise parameter threshold correspondingly included in an anaerobic exercise condition can be set. For example, the exercise parameter threshold correspondingly included in the aerobic exercise condition can be set to be the heart rate ranging from 120 to 150 beats per minute, and the exercise parameter threshold correspondingly included in the anaerobic exercise condition can be set to be the heart rate ranging from 170 to 180 per minute. Based on the aerobic exercise condition, by comparing the obtained exercise parameter of the user with the exercise parameter threshold included in the aerobic exercise condition, whether the exercise parameter satisfies the exercise parameter threshold can be determined. If the exercise parameter satisfies the exercise parameter threshold, the exercise parameter can be determined as satisfying the aerobic exercise condition. Then, the exercise state of the user can be determined as the aerobic exercise state. Based on the anaerobic exercise condition, by comparing the obtained exercise parameter of the user with the exercise parameter threshold included in the anaerobic exercise condition, whether the exercise parameter satisfies the exercise parameter threshold can be determined. If the exercise parameter satisfies the exercise parameter threshold, the exercise parameter can be determined to satisfy the anaerobic exercise condition. Then, the exercise state of the user can be determined as the anaerobic exercise state.

At S12, the current physical strength of the user is determined according to the exercise state.

In some embodiments, the physical strength can refer to energy used by the human body during activity, that is, an amount of energy that the body can continuously provide when the body continuously works at a largest strength. Under different exercise states, the consumption and recovery of the physical strength can be different for the user. The current physical strength of the user can be a sum of the physical strength consumption and the physical strength recovery. In some embodiments, according to the exercise state of the user, the physical strength consumption and the physical strength recovery of the user can be calculated during the exercise to determine the current physical strength of the user.

In some embodiments, FIG. 3 is a schematic flowchart showing determining the current physical strength of the user in the exercise training plan generation method according to some embodiments of the present disclosure. Determining the current physical strength of the user includes the following processes.

At S31, the physical strength condition of the user is determined according to the exercise state.

At S32, the current physical strength is determined according to the physical strength condition.

In some embodiments, the physical strength condition of the user can include the physical strength consumption condition of the user and the physical strength recovery condition of the user.

In some embodiments, by collecting historical exercise data of the user, a physical model of the user can be constructed according to the historical exercise data to determine the physical strength condition through the physical model.

In some embodiments, the physical strength condition of the user can be divided into an aerobic strength condition and an anaerobic strength condition. The aerobic strength condition can include an aerobic strength consumption condition and an aerobic strength recovery condition. The anaerobic strength condition can include an anaerobic strength consumption condition and an anaerobic strength recovery condition. In some embodiments, an aerobic strength consumption rate and an aerobic strength recovery rate of the user under the exercise state can be calculated according to the exercise parameter of the user through the physical model. The aerobic strength consumption condition and aerobic strength recovery condition of the user can be determined according to the aerobic strength consumption rate and the aerobic strength recovery rate. The anaerobic strength consumption rate and the anaerobic strength recovery rate of the user can be calculated under the exercise state according to the exercise parameter through the physical model. The anaerobic strength consumption condition and the anaerobic strength recovery condition of the user can then be determined according to the anaerobic strength consumption rate and the anaerobic strength recovery rate. Then, the current physical strength of the user can be determined based on the aerobic strength consumption condition, aerobic strength recovery condition, anaerobic strength consumption condition, and anaerobic strength recovery condition together. In some embodiments, the current physical strength of the user can include the current aerobic strength and current anaerobic strength of the user. The current aerobic strength of the user can be determined according to the aerobic strength consumption rate and the aerobic strength recovery rate of the user. The current anaerobic strength of the user can be determined according to the anaerobic strength consumption rate and the anaerobic strength recovery rate.

In some embodiments, the physical strength condition of the user can be divided into strength consumption condition and strength recovery condition. The anaerobic strength condition of the user can include the anaerobic strength consumption condition and/or the anaerobic strength recovery condition. The aerobic strength condition of the user can include the aerobic strength consumption condition and/or the aerobic strength recovery condition.

In some embodiments, when the exercise state of the user is the anaerobic exercise state, the anaerobic strength condition of the user can only include the anaerobic strength consumption condition. That is, during the anaerobic exercise, only the anaerobic strength for the user to perform the anaerobic exercise can be consumed, and the anaerobic strength may not be recovered. The aerobic strength condition of the user may not include the aerobic strength consumption condition and the aerobic strength recovery condition. That is, during the anaerobic exercise of the user, only the anaerobic strength for the user to perform the anaerobic exercise can be consumed, while the aerobic strength for the user to perform the aerobic exercise may not be consumed and recovered. In some embodiments, when the exercise state of the user is the anaerobic exercise state, the current physical strength of the user can be determined by calculating the anaerobic strength consumption of the user. The current anaerobic strength value of the user can be equal to an anaerobic strength value before the user performing the anaerobic exercise minus an anaerobic strength value consumed during the anaerobic exercise. The current aerobic strength value of the user can be an aerobic strength value before the user performing the anaerobic exercise. The current physical strength of the user can be a sum of the current anaerobic strength value and the current aerobic strength value.

In some embodiments, when the exercise state of the user is the aerobic exercise state, the anaerobic strength condition of the user can include the anaerobic strength recovery condition. That is, when the anaerobic strength of the user is consumed, and the user is performing the aerobic exercise, the anaerobic strength of the user may not be consumed, and the anaerobic strength of the user can be recovered. The aerobic strength condition of the user can only include the aerobic strength consumption condition. That is, during the aerobic exercise of the user, only the aerobic strength of the user can be consumed. In some embodiments, the current physical strength of the user can be determined by calculating the aerobic strength consumption and the anaerobic strength recovery. The current anaerobic strength value of the user can be a sum of the anaerobic strength value before the user performs the aerobic exercise and the anaerobic strength value recovered during the aerobic exercise. The current aerobic strength value of the user can be equal to the aerobic strength value before the user performs the aerobic exercise minus the aerobic strength value consumed during the aerobic exercise. The current physical strength of the user can be equal to the current anaerobic strength value and the current aerobic strength value of the user.

In some embodiments, the exercise state of the user can include a daily activity state. When the exercise state of the user is the daily activity state, the anaerobic strength of the user can include the anaerobic strength recovery condition. That is, when the anaerobic strength of the user is consumed before performing the daily activity, the anaerobic strength of the user may not be consumed when the user performs the daily activity, and the anaerobic strength of the user can be recovered. In some embodiments, when the anaerobic strength of the user is not consumed before the user performs the daily activity, and the user is performing the daily activity, the anaerobic strength of the user may not be consumed nor recovered. The aerobic strength condition of the user can include the aerobic strength recovery condition. That is, when the aerobic strength of the user is consumed before the user performs the daily activity, and when the user is performing the daily activity, the aerobic strength of the user may not be consumed, and the aerobic strength of the user can be recovered. In some embodiments, when the aerobic strength of the user is not consumed before the user performs the daily activity, and when the user is performing the daily activity, the aerobic strength of the user may not be consumed or recovered. In some embodiments, the current physical strength of the user can be determined by calculating the recovery of the aerobic strength of the user and the recovery of the anaerobic strength of the user. The current anaerobic strength value of the user can be a sum of the anaerobic strength value of the user before performing the daily activity and the anaerobic strength value recovered during the daily activity. The current aerobic strength value of the user can be a sum of the aerobic strength value before the user performs the daily activity and the aerobic strength value recovered during the daily activity. The current physical strength of the user can be equal to the sum of the current anaerobic strength value and the current aerobic strength value of the user.

In some embodiments, the physical state of the user can include at least one of a first physical strength condition of the user under the aerobic exercise state, a second physical strength condition of the user under the anaerobic exercise state, a third physical strength condition of the user under the daily activity state.

In some embodiments, the first physical strength condition of the user in the anaerobic exercise state can include a first physical strength consumption condition. That is, only the physical strength of the user can be consumed under the anaerobic exercise. In fact, during the anaerobic exercise, the anaerobic strength can be consumed, while the aerobic strength may not be consumed nor recovered. Thus, in some embodiments, the first physical strength consumption state can include a first physical strength consumption value, which can be the anaerobic strength consumption value of the user. The current physical strength of the user can be equal to the sum of the aerobic strength value and the anaerobic strength value before the anaerobic exercise minus the first physical strength consumption value.

In some embodiments, the second physical strength condition of the user in the aerobic exercise state can include the second physical strength consumption condition and the first physical strength recovery condition. That is, the aerobic strength can be consumed when the user is in the aerobic exercise state. When the user is switched from the anerobic exercise to the aerobic exercise, a certain anaerobic strength can be recovered. That is, the aerobic strength can be consumed and the anaerobic strength may not be consumed when the user performs the aerobic exercise. If the anaerobic exercise is performed before the aerobic exercise is performed, that is the anaerobic strength is consumed, after the user is switched to the aerobic exercise state, the anaerobic strength can be recovered. In some embodiments, the second physical strength consumption condition can include the second physical consumption value, i.e., the aerobic strength consumption value of the user, and the first physical strength recovery condition can include the first physical strength recovery value, i.e., the anaerobic strength recovery value of the user. The current physical strength of the user can be equal to the sum of the aerobic strength value and the anaerobic strength value before the aerobic exercise minus the second physical strength consumption value and plus the first physical strength recovery value.

In some embodiments, the third physical strength condition under the daily activity state can include the second physical strength recovery condition. That is, under the daily activity state, the physical strength may not be consumed, and the physical strength can be recovered. In some embodiments, during the daily activity, the aerobic strength and the anaerobic strength may not be consumed. When the anaerobic exercise is performed before the daily activity, the anaerobic strength can be consumed. After switching to the daily activity state, the anaerobic strength can be recovered. If the aerobic exercise is performed before the daily activity, the aerobic strength can be consumed. After switching to the daily activity state, the aerobic strength can be recovered. If the aerobic strength and the anaerobic strength are consumed during the daily activity, the aerobic strength and the anaerobic strength can be recovered. In some embodiments, the second physical strength recovery state can include the second physical strength recovery value, i.e., the sum of the aerobic strength recovery value and the anaerobic strength recovery value. The current physical value of the user can be the sum of the aerobic strength value and the anaerobic strength value before the daily activity and plus the second physical strength recovery value.

In some embodiments, when the anaerobic strength has recovered to the preset anaerobic strength threshold, the recovery of anaerobic strength may stop.

In some embodiments, when aerobic strength has recovered to the preset aerobic strength threshold, the recovery of aerobic strength may stop.

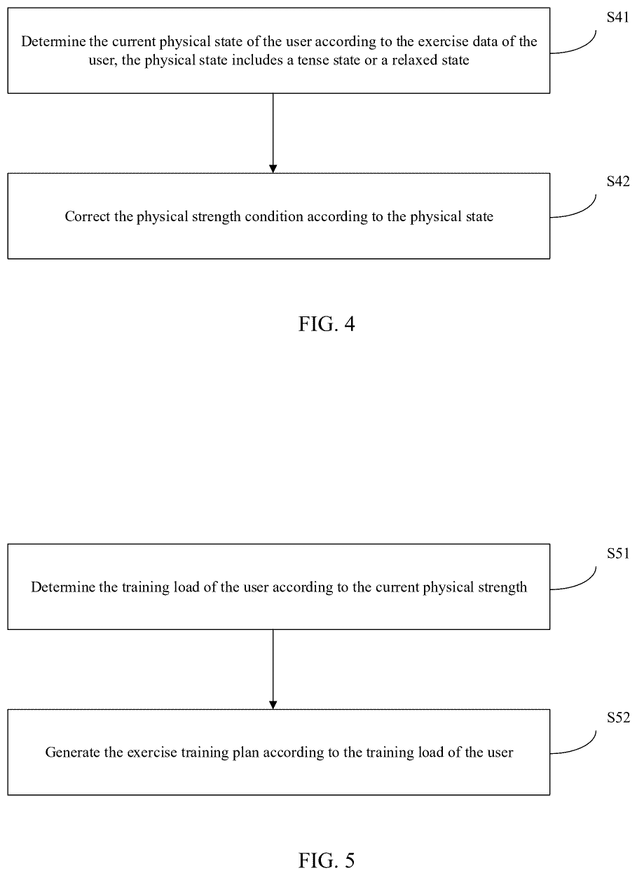

In some embodiments, FIG. 4 is a schematic flowchart of adjusting the physical strength condition in the exercise training plan generation method according to some embodiments of the present disclosure.

At S41, the current physical state of the user is determined according to the exercise data of the user, the physical state includes a tense state or a relaxed state.

At S42, the physical strength condition is adjusted according to the physical state.

In some embodiments, the physical strength recovery condition can be closely related to the physical state of the body. The physical state can be divided into the tense state and the relaxed state. In different physical states, the physical strength consumption and recovery of the user can be different. In the relaxed state, the heart rate can be low, the heart rate variability (HRV) index can be high, and the physical strength recovery rate can be high. In the tense state, the heart rate can be low, the HRV index can be low, and the physical strength recovery rate can be low.

In some embodiments, under different physical states, the physical strength consumption and recovery of the body can be different. For example, the physical strength can be consumed faster in the tense state than in the relaxed state, and/or the physical strength can be recovered slower in the tense state than in the relaxed state.

In some embodiments, the current exercise data of the user can be obtained. The exercise data can include the current heart rate data and heart rate variability data of the user. Then, the current physical state of the user can be determined according to the exercise data of the user. For different physical states, corresponding strength condition adjustment coefficients can be set. That is, the tense state can correspond to a strength condition adjustment coefficient, and the relaxed state can correspond to a strength condition adjustment coefficient. After the physical state of the user is determined, the physical strength condition can be adjusted according to the physical strength condition adjustment coefficient corresponding to the physical state.

In some embodiments, the physical strength condition adjustment coefficient corresponding to the tense state can be smaller than the physical strength condition adjustment coefficient corresponding to the relaxed state.

In some embodiments, the physical strength condition adjustment coefficient corresponding to the tense state can be less than 1, while the physical strength condition adjustment coefficient corresponding to the relaxed state can be greater than 1.

In some embodiments, the heart rate data threshold and the heart rate variability threshold used to determine the current physical state of the user can be preset. By comparing the current heart rate data to the heart rate data threshold and/or the current heart rate variability data to the heart rate variability threshold, the current physical state of the user can be determined according to the comparison result. In some embodiments, when the current heart rate data of the user is smaller than or equal to the heart rate data threshold and/or the current heart rate variability data of the user is smaller than or equal to the heart rate variability threshold, the physical state of the user can be determined as the relaxed state. When the current heart rate data of the user is greater than the heart rate data threshold and/or the current heart rate variability data is greater than the heart rate variability threshold, the physical state of the user can be determined to be the tense state.

At S13, the exercise training plan is generated according to the current physical strength.

In some embodiments, the current physical strength of the user can be used to guide the user in exercise training. In some embodiments, after the current physical strength of the user is determined, through the manual setting of an exercise coach or system automated recommendation, the exercise training plan suitable for the user can be generated according to the current physical strength of the user.

In some embodiments, FIG. 5 is a schematic flowchart of generating the exercise training plan in the exercise training plan generation method according to some embodiments of the present disclosure. Generating the exercise training plan includes the following processes.

At S51, the training load of the user is determined according to the current physical strength.

At S52, the exercise training plan is generated according to the training load of the user.

In some embodiments, the training load can include a training intensity and a training amount. Under the same training load, the greater the training intensity is, the smaller the training amount is. For example, the training load for running can be represented by running time length and running speed. Under the same training load, the greater the running speed is, the shorter the running duration. In some embodiments, the training load of the user can be calculated according to the current physical strength of the user. Then, the exercise training plan can be generated according to the training load. The generated exercise training plan can include at least one exercise item. The total load of the at least one exercise item can match the training load of the user. In some embodiments, the total load of at least one exercise item matching the training load of the user can be represented that the total load of all the exercise types can approximately equal to the training load of the user.

In some embodiments, the exercise items can include aerobic exercises such as yoga, walking, jogging, ice skating, swimming, cycling, Tai Chi, aerobics, and rhythmic gymnastics, as well as anaerobic exercises such as sprinting, weightlifting, throwing, high jump, long jump, tug of war, push-ups, diving, and strength training.

In some embodiments, when determining whether the total load of at least one exercise item matches the training load of the user, a difference between the total load of the at least one exercise item and the training load of the user can be calculated according to the total load of the at least one exercise item and the training load of the user. Then, the calculated difference can be compared to the preset difference. When the difference is smaller than or equal to the preset difference, the total load of the at least one exercise item can be determined to match the training load. For example, the load corresponding to the jogging in the exercise item for an hour can be 600 calories, the load corresponding to swimming for an hour can be 650 calories, and the load corresponding to yoga for an hour can be 360 calories. Assume that the training load of the user can be determined to be 500 calories according to the current strength. Assume that the preset difference is 15 calories. The generated exercise training plan can be jogging for 50 minutes. The load corresponding to jogging for 50 minutes can be 500 calories, which has a zero difference from the training load of 500 calories of the user. Thus, the total load of the exercise item can be determined as matching the training load. The generated exercise training plan can be swimming for 45 minutes. The load corresponding to swimming for 45 minutes can be 487.5 calories, which differs from the training load 500 calories of the user by 12.5 calories. Since 12.5 calories is smaller than the preset difference of 15 calories, the total load of the exercise can be determined as matching the training load. Generated exercise training plan can include jogging for 30 minutes and yoga for 30 minutes. The load corresponding to jogging for 30 minutes can be 325 calories, and the load corresponding to yoga for 30 minutes can be 180 calories. The total load can be 505 calories, which differs from the training load 500 calories of the user by 5 calories. Since 5 calories is smaller than the preset difference of 15 calories, the total load of the exercise can be determined as matching the training load.

In some embodiments, since the anaerobic strength can recover during exercise when the body performs the anaerobic exercise, the physical strength condition of the user can be determined according to the exercise parameter of the user. The training load of the user can be determined according to the current physical strength and the physical strength condition of the user. The physical strength condition can be represented as the physical strength consumption condition of the user and/or the physical strength recovery condition of the user. For example, the load corresponding to muscle strength training for an hour of the exercise can be 300 calories. During the anaerobic strength recovery rate can be 100 calories per hour when the user is in the anaerobic exercise state. Assume that the training load of the user can be determined to be 400 calories according to the current physical strength of the user, the exercise training plan can be muscle strength training for 2 hours when the exercise state of the user is in the anaerobic exercise state. The training load of the user can be a sum of the 400 calories determined by the current physical strength and the 200 calories of the anaerobic strength recovered during 2 hours in the anaerobic exercise state, i.e., the training load is 600 calories. Correspondingly, the load corresponding to the muscle strength training for 2 hours in the exercise training plan can be 600 calories, which equals the training load of 600 calories of the user. Thus, the total load of the exercise item can be determined as matching the training load.

In some embodiments, an electronic device, such as a wearable device, a computer, or a smartphone can be configured or controlled to operate according to the generated training plan so that the electronic device has an improved function and is technically more advanced for guiding the user through the training process.

Based on the above, in the exercise training plan generation method of embodiments of the present disclosure, the exercise state of the user can be obtained. The exercise state can include any one of an aerobic exercise state and an anaerobic exercise state. The current physical strength of the user can be determined according to the exercise state to generate the exercise training plan according to the current physical strength. The current physical strength of the user can be accurately obtained based on different exercise states of the user. Further, the exercise training plan can be generated based on the current physical strength of the user, which facilitates the effective training and daily physical strength management.

In some embodiments, the exercise training plan generation method can be applied to an electronic device. The exercise training plan can be generated through the electronic device. In some embodiments, the electronic device can further include a display apparatus. The electronic device can control the display apparatus to display an image interface corresponding to the current physical strength when the exercise training plan is generated. In some embodiments, the display apparatus can be pre-designed to display the image interface of the current physical strength to control the display apparatus to display the current physical strength in the image interface.

In some embodiments, an image assembly of a physical strength progress bar and a physical strength filling block representing the magnitude of the current physical strength can be generated in the image interface. Based on the image assembly, the display apparatus can be controlled to display the physical strength progress bar and the physical strength filling block representing the magnitude of the current physical strength. In some embodiments, the size and shape of the current physical strength filling block can be determined according to the current physical strength of the user to generate the image assembly matching the size and shape of the current physical strength filling block. Moreover, the image assembly representing the physical strength progress bar can be generated according to the preset size and the shape. The image assembly matching the size and shape of the current physical strength filling block and the image assembly representing the physical strength progress bar can be superimposed and displayed in the image interface. Thus, the display apparatus can be controlled to superimpose and display the physical strength progress bar and the current physical strength filling block in the image interface according to the size and shape of the current physical strength filling block.

In some embodiments, the image assembly of the physical strength progress bar can be fixed on the image interface. The size and shape of the physical strength filling block can be determined according to the current physical strength to generate the image assembly matching the size and shape of the current physical strength filling block to further control the display apparatus to superimpose and display the image assembly matching the size and shape of the current physical strength filling block on the image assembly of the physical strength progress bar fixed in the image interface. Thus, the display apparatus can be controlled to superimpose and display the physical strength filling block on the physical strength progress bar according to the size and shape of the physical strength filling block to display the magnitude of the current physical strength on the image interface.

In some embodiments, the display apparatus can be controlled to display two physical strength progress bars on the image interface, including one aerobic strength progress bar configured to display the current aerobic strength value of the user, and an anaerobic strength progress bar configured to display the current anaerobic strength value of the user. When the display apparatus is controlled to display the aerobic strength progress bar in the image interface, the size and shape of the aerobic strength filling block can be determined according to the current aerobic strength value to generate the image assembly matching the size and shape of the aerobic strength filling block. Moreover, the image assembly representing the aerobic strength progress bar can be generated according to the preset size and shape to superimpose and display the image assembly matching the size and shape of the aerobic strength filling block and the image assembly representing the aerobic progress bar in the image interface to display the size of the current aerobic physical strength in the image interface. When the display apparatus is controlled to display the anaerobic strength progress bar in the image interface, the size and shape of the anaerobic strength filling block can be determined according to the current anaerobic strength value of the user to generate the image assembly matching the size and shape of the anaerobic strength filling block. Moreover, the image assembly representing the anaerobic strength progress bar can be generated according to the preset size and shape to superimpose and display the image assembly matching the size and shape of the anaerobic strength filling block and the image assembly representing the anaerobic strength progress bar in the image interface to display the size of the current anaerobic strength in the image interface.

In some embodiments, when the display apparatus is controlled to display two physical strength progress bars in the image interface, the image assembly of the two physical strength progress bars can be fixed in the image interface. The size and shape of the aerobic strength filling block can be determined according to the current physical strength to generate the image assembly matching the size and shape of the aerobic strength filling block. Moreover, the display apparatus can be controlled to superimpose and display the image assembly matching the size and shape of the aerobic strength filling block and the image assembly of the aerobic strength progress bar fixed in the image interface. Thus, the size of the current aerobic strength can be displayed in the image interface. By determining the size and shape of the anaerobic strength filling block according to the current anaerobic strength, the image assembly matching the size and shape of the anaerobic strength filling block can be generated. Further, the display apparatus can be controlled to impose and display the image assembly matching the size and shape of the anaerobic strength filling block and the image assembly of the anaerobic strength progress bar fixed in the image interface to display the size of the current anaerobic strength in the image interface.

In some embodiments, the display apparatus can also be controlled to display a percentage value of the current physical strength. In some embodiments, the percentage value of the current physical strength can be determined according to the current physical strength and the largest physical strength by obtaining the largest physical strength of the user. Moreover, the display apparatus can be controlled to impose and display the percentage value of the current physical strength in the image interface according to the percentage value of the current physical strength.

In some embodiments, the display apparatus can be controlled to display the value corresponding to the current physical strength. In some embodiments, the value corresponding to the current physical strength can be determined according to the current physical strength to control the display apparatus to impose and display the value corresponding to the current physical strength in the image interface.

In some embodiments, the display apparatus can be controlled to display the mark related to the fatigue level of the user. In some embodiments, the fatigue level of the user can be determined according to the current physical strength. The mark corresponding to the fatigue level can be determined according to the fatigue level. Further, the display apparatus can be controlled to superimpose and display the mark corresponding to the fatigue level in the image interface.

In some embodiments, the display apparatus can also be controlled to display the training load of the user. In some embodiments, the training load can be determined according to the current physical strength to control the display apparatus to superimpose and display the training load in the image interface.

The sequence numbers of the processes of embodiments of the present disclosure do not imply the execution order. The execution order of the processes can be determined according to the functions and internal logic, which do not form any limitation on embodiments of the present disclosure.

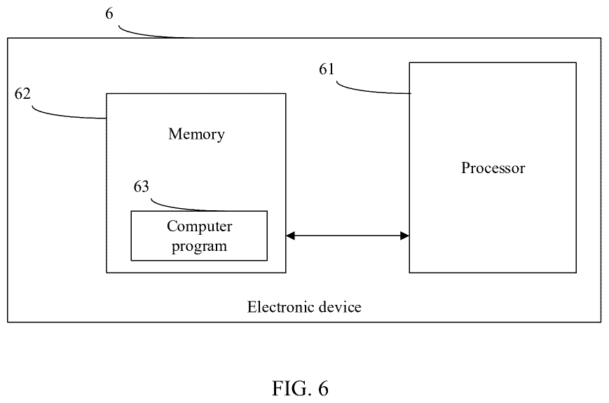

In embodiments of the present disclosure, FIG. 6 is a schematic structural block diagram of an electronic device 6 according to some embodiments of the present disclosure. As shown in FIG. 6, the electronic device 6 includes at least one processor 61, at least one memory 62, and at least one computer program 63, e.g., the program of the exercise training plan generation method, stored in the at least one memory 62 and can run on the at least one processor 61. When the at least one processor 61 executes the at least one computer program 63, the processes of the exercise training generation method can be realized. For the detailed description, reference can be made to related description of embodiments of the present disclosure, which is not repeated.

For example, the at least one computer program 63 can be divided into one or more modules (units), which are stored in the at least one memory 62 and executed by the at least one processor 61 to implement the present disclosure. The one or more modules can be a series of computer program instruction segments that can realize specific functions. The instruction segments can be used to describe the execution process of the at least one computer program 63 in the electronic device 6.

The electronic device includes, but is not limited to, the at least one processor 61 and the at least one memory 62. Those skilled in the art can understand that FIG. 6 shows merely an example of the electronic device 6 and does not limit the electronic device 6. The electronic device 6 can include more or fewer components than the electronic device in the drawings, or different components. In some other embodiments, some units can be grouped. For example, the electronic device can include an input/output device, a network access device, a bus.

The at least one processor 61 can be a central processing unit (CPU), other general-purpose processors, a Digital Signal Processors (DSP), an Application Specific Integrated Circuits (ASIC), a Field-Programmable Gate Arrays (FPGA), or other programmable logic devices, discrete gate or transistor logic devices, discrete hardware components, etc. The general-purpose processor can be a microprocessor or any conventional processor.

The at least one memory 62 can be the internal storage unit of the electronic device 6, e.g., a hard drive or memory of the electronic device 6. The at least one memory 62 can also be an external storage device of the electronic device 6, e.g., a plug-in hard drive, a Smart Media Card (SMC), a Secure Digital (SD) card, and a Flash Card arranged at the electronic device 6. Furthermore, the memory 62 can include internal storage units and external storage devices of the electronic device 6. The at least one memory 62 can be used to store the computer program and other programs and data required by the electronic device. The at least one memory 62 can also be used to temporarily store data that has been output or will be output.

Since the information interaction and execution process between the apparatus/units are based on the same concept of embodiments of the present disclosure, for the technical effects brought by the specific functions, reference can be made to method embodiments, which are not repeated here.

Embodiments of the present disclosure also provide a computer-readable storage medium. The computer-readable storage medium can store a computer program that, when executed by the processor, causes the processor to realize the processes of method embodiments. In some embodiments, the computer-readable storage medium can be non-volatile or volatile.

Embodiments of the present disclosure provide a computer program product. When the computer program product is running at a mobile terminal, the mobile terminal can realize processes of method embodiments when the mobile terminal executes the computer program.

Those skilled in the art can clearly understand that the division of the above functional units and modules is merely exemplary for the sake of convenience and brevity in the description. The above functions can be assigned to different functional units or modules in practical applications as needed. That is, the internal structure of the apparatus can be divided into different functional units or modules to implement all or a part of the functions described above. The functional units or modules of embodiments of the present disclosure can be integrated into a processing unit or can exist separately as individual units, or two or more units can be integrated into one unit. The integrated units can be implemented in the form of hardware or in the form of software functional units. In addition, the specific names of the functional units or modules are merely used to facilitate distinguishing the functional units and modules from each other and are not used to limit the scope of the present disclosure. For the operation processes of the units and modules of the above system, reference can be made to corresponding processes in method embodiments, which are not repeated here.

If the integrated modules/units are implemented in the form of software functional units and sold or used as independent products, the integrated modules/units can be stored in a computer-readable storage medium. Based on this understanding, all or a part of the processes in the method embodiments described above can also be implemented by instructing the relevant hardware through a computer program. The computer program can be stored in a computer-readable storage medium that, when executed by the processor, causes the processor to implement the processes of the method embodiments described above. The computer program can include computer program codes, which can be in the form of source codes, object codes, executable files, or some intermediate forms. The computer-readable medium can include any entity or apparatus capable of carrying the computer program codes, such as recording media, USB drives, portable hard drives, magnetic disks, optical disks, computer memory, Read-Only Memory (ROM), Random Access Memory (RAM), electrical carrier signals, telecommunication signals, software distribution media, etc. The contents included in the computer-readable medium can be appropriately increased or decreased according to the requirements of legislation and patent practices in jurisdictions. For example, in some jurisdictions, according to legislation and patent practices, the computer-readable medium does not include electrical carrier signals and telecommunication signals.

Embodiments of the present disclosure can focus differently on description. For the part not detailed or recorded in an embodiment, reference can be made to the related description of the embodiments.

Embodiments of the present disclosure are merely used to describe the technical solution of the present disclosure, not to limit the present disclosure. Although the present disclosure is described in detail with reference to embodiments of the present disclosure, those skilled in the art should understand that modifications can still be made to the technical solution in embodiments of the present disclosure or equivalent replacements can be performed on some technical features. These modifications or replacements do not cause the essence of the corresponding technical solution to depart from the spirit and scope of the technical solutions of embodiments of the present disclosure and should be within the scope of the present disclosure.

What is claimed is:

1. A method comprising:
obtaining an exercise state of a user, the exercise state including at least one of an aerobic exercise state or an anaerobic exercise state;
determining a current physical strength of the user according to the exercise state; and
generating an exercise training plan according to the current physical strength.

2. The method according to claim 1, further comprising:
obtaining an exercise parameter of the user;
wherein obtaining the exercise state includes:
determining the exercise state according to the exercise parameter.

3. The method according to claim 2, wherein determining the exercise state according to the exercise parameter includes:
determining whether the exercise parameter satisfies an aerobic exercise condition or an anaerobic exercise condition to obtain a determination result; and
determining whether the exercise state is the aerobic exercise state or the anaerobic exercise state according to the determination result, including:
in response to the exercise parameter satisfying the aerobic exercise condition, determining that the exercise state is the aerobic exercise state; or
in response to the exercise parameter satisfying the anaerobic exercise condition, determining that the exercise state is the anaerobic exercise state.

4. The method according to claim 1, further comprising:
determining a physical strength condition of the user according to the exercise state;
wherein:
the physical strength condition represents at least one of a physical strength consumption condition or a physical strength recovery condition of the user; and
determining the current physical strength according to the exercise state includes:
determining the current physical strength according to the physical strength condition.

5. The method according to claim 4, wherein:
the physical strength condition includes at least one of a first physical strength condition of the user in the aerobic exercise state or a second physical strength condition of the user in the anaerobic exercise state; and
determining the current physical strength according to the physical strength condition includes at least one of:
in response to the exercise state being the aerobic exercise state, determining the current physical strength according to the first physical strength condition; or
in response to the exercise state being the anaerobic exercise state, determining the current physical strength according to the second physical strength condition.

6. The method according to claim 4, wherein:
the exercise state further includes a daily activity state;
the physical strength condition includes a physical strength condition in the daily activity state; and
determining the current physical strength according to the physical strength condition includes:
in response to the exercise state being the daily activity state, determining the current physical strength according to the physical strength condition in the daily activity state.

7. The method according to claim 4, further comprising:
determining a physical state of the user according to exercise data of the user, the physical state including a tense state or a relaxed state; and
adjusting the physical strength condition according to the physical state.

8. The method according to claim 1, wherein:
generating the exercise training plan according to the current physical strength includes:
determining a training load of the user according to the current physical strength; and
generating the exercise training plan according to the training load; and
the exercise training plan includes at least one exercise item, and a total load of the at least one exercise item matches the training load of the user.

9. The method according to claim 8, further comprising:
obtaining the total load of the at least one exercise item; and
determining whether the total load of the at least one exercise item matches the training load;
wherein generating the exercise training plan includes:
generating the exercise training plan according to the training load in response to the total load of the at least one exercise item matching the training load.

10. The method according to claim 9, further comprising:
determining a difference between the total load and the training load;
wherein determining whether the total load matches the training load includes:
determining that the total load matches the training load in response to the difference being less than or equal to a preset difference.

11. The method according to claim 8, further comprising:
determining a physical strength condition of the user according to exercise data of the user, the physical strength condition representing at least one of a physical consumption condition or a physical recovery condition of the user;
wherein determining the training load includes:
determining the training load of the user according to the current physical strength and the physical strength condition.

12. The method according to claim 1, further comprising:
controlling a display apparatus to display an image interface corresponding to the current physical strength.

13. The method according to claim 12, further comprising:
controlling the display apparatus to display the current physical strength on the image interface.

14. The method according to claim 13, wherein controlling the display apparatus to display the current physical strength on the image interface includes:
determining a size and a shape of a physical strength filling block according to the current physical strength, the physical strength filling block representing a magnitude of the current physical strength; and
controlling the display apparatus to:
superimpose and display a physical strength progress bar and the physical strength filling block on the graphical interface according to the size and the shape of the physical strength filling block; or
superimpose and display the physical strength filling block on the physical strength progress bar according to the size and shape of the physical strength filling block.

15. The method according to claim 12, further comprising:
obtaining maximum physical strength of the user; and
determining a percentage value of the current physical strength according to the current physical strength and the maximum physical strength;
wherein controlling the display apparatus to display the current physical strength on the image interface includes:
controlling the display apparatus to superimpose and display the percentage value of the current physical strength on the image interface.

16. The method according to claim 12, further comprising:
determining a value corresponding to the current physical strength; and
controlling the display apparatus to superimpose and display the value corresponding to the current physical strength on the image interface.

17. The method according to claim 12, further comprising:
determining a fatigue level of the user according to the current physical strength;
determining a marker corresponding to the fatigue level; and
controlling the display apparatus to superimpose and display the marker on the image interface.

18. The method according to claim 12, further comprising:
determining a training load of the user according to the current physical strength; and
controlling the display apparatus to superimpose and display the training load on the image interface.

19. An electronic device comprising:
at least one processor; and
at least one memory storing at least one computer program that, when executed by the at least one processor, causes the at least one processor to:
obtain an exercise state of a user, the exercise state including at least one of an aerobic exercise state or an anaerobic exercise state;
determine a current physical strength of the user according to the exercise state; and
generate an exercise training plan according to the current physical strength.

20. A computer-readable storage medium storing at least one computer program that, when executed by at least one processor, causes the at least one processor to:
obtain an exercise state of a user, the exercise state including at least one of an aerobic exercise state or an anaerobic exercise state;
determine a current physical strength of the user according to the exercise state; and
generate an exercise training plan according to the current physical strength.
